(19) United States
(12) Patent Application Publication
(10) Pub. No.: US 2010/0249394 A1
(43) Pub. Date: Sep. 30, 2010

(54) PROCESSES FOR PRODUCING DECITABINE

(75) Inventors: Yunqi Liu, Baton Rouge, LA (US); Gregory H. Lambeth, Baton Rouge, LA (US); Brian W. Nixon, Greenwell Springs, LA (US)

Correspondence Address: ALBEMARLE CORPORATION;PATENT DEPARTMENT, 451 FLORIDA STREET, BATON ROUGE, LA, 70801, US

(73) Assignee: Albemarle Corporation, Baton Rouge, LA (US)

(21) Appl. No.: 12/731,760

(22) Filed: Mar. 25, 2010

Related U.S. Application Data
(60) Provisional application No. 61/163,704, filed on Mar. 26, 2009.

(51) Int. Cl. C07H 19/12

(52) U.S. Cl. 536/28.3

(57) ABSTRACT

New processes for producing decitabine are provided.

### BACKGROUND

Decitabine is an antimetabolite and demethylation agent that is used, e.g., in the treatment of Myelodysplastic Syndromes. The IUPAC name for decitabine is 4-amino-1-(2-deoxy-beta-D-erythro-pentofuranosyl)-1,3,5-triazin-2(1H)-one.

Due to their commercial availability, 5-azacytosine and protected 2-deoxy-D-ribose and are often used as the starting materials for the synthesis of decitabine.

For 2-deoxy-D-ribose, different leaving groups at the anomeric position such as chloride, acetate, and methoxy are often used for the coupling step when preparing decitabine. Different protecting groups such as acetyl, F-Moc, toluoyl, benzyl, or alkyl, are typically used to protect the hydroxyl groups on the 2-deoxy-D-ribose ring. Unlike the synthesis of azacitidine where a protected hydroxyl group at the 2-position helps to yield predominantly the beta anomer, the synthesis of decitabine is more challenging because of the lack of participation from the protected hydroxyl groups. Thus, in most cases when decitabine is synthesized using protected 2-deoxy-D-ribose, a complex mixture consisting of essentially equal amounts of alpha and beta anomers of the intermediate 4-amino-1-[3,5-di-O-(p-chlorobenzoyl)]-2-deoxy-D-ribofuranosyl-1,3,5-triazin-2(1H)-one, and many undesired side products, is obtained. A purification procedure such as column chromatography is often used to purify such a complex mixture to enrich the desired beta anomer coupling product before proceeding to the deprotection step. Such processes are used routinely in the lab. However, for large scale production, use of such a purification procedure tends to be inefficient, time consuming, and not suitable for commercial use.

Thus, there is a need for more efficient and scalable processes for the synthesis of decitabine, and in particular, for such processes that eliminate the need for purification of intermediates during the synthesis.

### THE INVENTION

This invention meets the above-described needs by providing new processes for producing decitabine, which processes require minimal or no purification of intermediates and provide commercially acceptable yields. Therefore, processes of this invention are particularly advantageous for large scale commercial production of decitabine.

Processes of this invention can comprise yielding at least 1-[3,5-di-O-(p-chlorobenzoyl)]-2-deoxy-alpha-D-ribofuranosyl-chloride in accordance with this invention, yielding beta:alpha anomers of 4-amino-1-[3,5-di-O-(p-chlorobenzoyl)]-2-deoxy-D-ribofuranosyl-1,3,5-triazin-2(1H)-one from at least the 1-[3,5-di-O-(p-chlorobenzoy1)]-2-deoxy-alpha-D-ribofuranosyl-chloride, and yielding decitabine from at least the beta:alpha anomers of 4-amino-1-[3,5-di-O-(p-chlorobenzoyl)]-2-deoxy-D-ribofuranosyl-1,3,5-triazin-2(1H)-one.

### Solvents Used In Processes of This Invention

Solvent A as used in processes of this invention comprises dioxane, toluene, or the like.

Solvent B as used in processes of this invention comprises acetonitrile, 1,2-dichloroethane, chloroform, dichloromethane, ethylacetate, toluene, alkanes, or other halogenated solvents.

Solvent C as used in processes of this invention comprises 1,2-dichloroethane, chloroform, dichloromethane, ethylacetate, or other halogenated solvents.

Suitable final rinse solvents as used in processes of this invention comprise methanol, ethanol, ethyl acetate, isopropanol, or the like.

### Catalysts Used In Processes of This Invention

Catalyst A as used in processes of this invention comprises 4-substituted pyridines such as 4-dimethylaminopyridine, 4-pyrrolidinopyridine, or other suitable 4-substituted pyridines.

Catalyst B as used in processes of this invention comprises chlorotrimethylsilane or ammonium sulfate.

Catalyst C as used in processes of this invention comprises trimethylsilyl trifluoromethanesulfonate, Tin (IV) chloride, or other Lewis acids.

### Inert Gas Used In Processes of This Invention

Inert gas as used in processes of this invention comprises nitrogen, argon, helium, or the like.

### Yielding At Least 1-[3,5-di-O-(p-chlorobenzoyl)]-2-deoxy-alpha-D-ribofuranosyl-chloride

Yielding at least 1-[3,5-di-O-(p-chlorobenzoyl)]-2-deoxy-alpha-D-ribofuranosyl-chloride in accordance with this invention can comprise the following:

Combining at least 2-deoxy-D-ribose and methanol to yield a first combination, wherein the amount of methanol is at least about 4 grams per gram of the 2-deoxy-D-ribose and the methanol is substantially anhydrous. By “substantially anhydrous” is meant methanol that contains essentially no water. Commercially available substantially anhydrous methanol typically contains about 0.002 wt % water, or less. In processes of this invention, the amount of methanol can be at least about 4 to about 15 grams per gram of the 2-deoxy-D-ribose.

Cooling the first combination to about 25° C. to about −20° C., by means familiar to those skilled in the art.

Combining at least an acyl halide and at least a portion of the first combination to yield a second combination. The second combination can be a solution. Suitable acyl halides can comprise acetyl chloride. At least a portion of the acyl halide reacts with the methanol to yield HCl in situ in the second combination. The amount of acyl halide is at least enough for generating in the second combination about 0.1 wt % to about 1 wt % HCl based on the amount of the methanol.

Maintaining the second combination at about 25° C. to about −25° C. for about 30 minutes to about 240 minutes, for example, for about 1 to about 2 hours.

Combining at least a first volatile tertiary amine and at least a portion of the second combination to yield a first product comprising 1-O-methyl-2-deoxy-D-ribofuranose and methanol. As used herein, volatile tertiary amines comprise volatile tertiary amines such as triethylamine, or the like. The amount of the first volatile tertiary amine can be at least about 1 molar equivalent based on the amount of the acyl halide. The first product can be a liquid.

Removing substantially all of the methanol from the first product, yielding a methanol-free first product. As used in this regard, removing “substantially all” of the methanol means removing an adequate amount of the methanol such that the methanol-free first product can be used in processes of this invention without detrimental effects, as will be familiar to those skilled in the art, and includes removing all of the methanol. Thus, a methanol-free first product is a first product from which substantially all of the methanol has been removed.

Combining at least a substantially anhydrous Solvent A, a Catalyst A, a second volatile tertiary amine, and at least a portion of the methanol-free first product to yield a third combination. The third combination can be a solution. As used herein, Solvent A is a solvent in which at least a substantial portion (e.g., about 50%) of the methanol-free first product remains in solution, but in which at least a substantial portion of an amine HCl salt does not remain in solution. The amount of the Solvent A can be at least about 1 to about 2 liters for every 100 grams of the 2-deoxy-D-ribose initially added in processes of this invention. The amount of the second volatile tertiary amine can be adequate to establish about 1:2 to about 1:2.2 moles of initially added 2-deoxy-D-ribose.

Combining at least p-chlorobenzoyl chloride and at least a portion of the third combination to yield a fourth combination, e.g., while maintaining the fourth combination at about 10° C. to about 40° C. The fourth combination can be a slurry. The amount of p-chlorobenzoyl chloride can be adequate to establish about 1:2 to about 1:2.2 moles of initially added 2-deoxy-D-ribose:p-chlorobenzoyl chloride.

Stirring the fourth combination for about 2 hours to about 6 hours at about 10° C. to about 40° C.

Filtering the fourth combination to yield a filtrate and a filter cake.

Collecting the filtrate.

Rinsing the filter cake with at least a substantially anhydrous rinsing solvent, such as dioxane, and recovering at least a portion of the rinsing solvent, i.e., the used rinsing solvent. Suitable rinsing solvents can comprise dioxane, toluene, acetone, and the like.

Optionally, concentrating the used rinsing solvent by removing some or substantially all of the Solvent A, e.g., the dioxane; or, for example, by removing some or substantially all of the Solvent A from the used rinsing solvent and then adding chloroform or a chloroform/acetic acid system to the remaining used rinsing solvent.

Combining at least acetic acid, at least a portion of the filtrate, and at least a portion of the used rinsing solvent (or concentrated used rinsing solvent), and optionally, a suitable drying agent, e.g., acetyl chloride, to yield a fifth combination. The fifth combination can be a solution. The amount of the used rinsing solvent can be adequate to establish about 5-30 wt % of used rinsing solvent based on the total weight of the initially added 2-deoxy-D-ribose and the added used rinsing solvent. A suitable drying agent can comprise acetyl chloride, propionyl chloride, butyryl chloride, and the like. The amount of the acetic acid can be adequate to establish about 2:1 to about 1:2 of added Solvent A:acetic acid. When used, the amount of suitable drying agent, e.g., acetyl chloride, can be adequate to establish more than about 1 gram of the suitable drying agent per 100 ml of the acetic acid.

Combining at least HCl gas and at least a portion of the fifth combination at about −10° C. to about 25° C. to yield a sixth combination. The sixth combination can be a slurry. The amount of HCl gas can be adequate to establish at least about 1:4 to about 1:8 moles of initially added 2-deoxy-D-ribose:HCl gas.

Stirring the sixth combination. For example, stirring the sixth combination for at least about 5 minutes. Optionally, adding at least an alkane, such as hexane or hexanes, to the sixth combination.

Collecting a second product comprising 1-[3,5-di-O-(p-chlorobenzoyl)]-2-deoxy-alpha-D-ribofuranosyl-chloride from the sixth combination. For example, the second product can be collected from the sixth combination by vacuum filtration. Optionally, rinsing the second product with at least an alkane, such as hexane or hexanes; e.g., with enough of the hexane or hexanes for an efficient rinse.

Drying the second product, e.g., by vacuum-drying at ambient temperature, or by other means familiar to those skilled in the art, to yield at least 1-[3,5-di-O-(p-chlorobenzoyl)]-2-deoxy-alpha-D-ribofuranosyl-chloride. Even though predominantly 1-[3,5-di-O-(p-chlorobenzoyl)]-2-deoxy-alpha-D-ribofuranosyl-chloride is yielded, 1-[3,5-di-O-(p-chlorobenzoyl)]-2-deoxy-beta-D-ribofuranosyl-chloride is also yielded. As used herein, “drying” means removing a substantial portion, substantially all, or all volatiles.

Yielding at least 1-[3,5-di-O-(p-chlorobenzoyl)]-2-deoxy-alpha-D-ribofuranosyl-chloride in accordance with this invention can comprise the following:

Combining at least 1-O-methyl-3, 5-di-O-p-chlorobenzoyl-2-deoxy-alpha/beta-D-ribose, acetic acid, an acyl halide, such as acetyl chloride, and a substantially anhydrous Solvent C to yield a first combination.

While stirring and in the presence of an inert gas, feeding gaseous HCl to the first combination at about −10° C. to about 25° C. to yield a second combination comprising 1-[3,5-di-O-(p-chlorobenzoyl)]-2-deoxy-alpha-D-ribofuranosyl-chloride. The second combination can be a slurry.

Stirring the second combination at about −10° C. to about 25° C. for at least about 5 minutes.

Yielding from the second combination a product comprising 1-[3,5-di-O-(p-chlorobenzoyl)]-2-deoxy-alpha-D-ribofuranosyl-chloride. The product can also comprise 1-[3,5-di-O-(p-chlorobenzoyl)]-2-deoxy-beta-D-ribofuranosyl-chloride.

Rinsing the product with a rinse comprising substantially anhydrous hexane or hexanes, hydrocarbons, toluene, or the like.

Drying the product to yield at least 1-[3,5-di-O-(p-chlorobenzoyl)]-2-deoxy-alpha-D-ribofuranosyl-chloride. Drying can be accomplished, e.g., by vacuum-drying at ambient temperature, or by other means familiar to those skilled in the art.

Yielding Beta:Alpha Anomers of 4-amino-1-[3,5-di-O-(p-chlorobenzoyl)]-2-deoxy-D-ribofuranosyl-1,3,5-triazin-2(1H)-one From At Least 1-[3,5-di-O-(p-chlorobenzoyl)]-2-deoxy-alpha-D-ribofuranosyl-chloride

Yielding beta:alpha anomers of 4-amino-1-[3,5-di-O-(p-chlorobenzoyl)]-2-deoxy-D-ribofuranosyl-1,3,5-triazin-2(1H)-one from at least 1-[3,5-di-O-(p-chlorobenzoyl)]-2-deoxy-alpha-D-ribofuranosyl-chloride in accordance with this invention can comprise the following:

Combining at least 5-azacytosine, hexamethyldisalazane (“HMDS”) and a Catalyst B in the presence of an essentially dry first inert gas to yield a first intermediate product comprising 2-[(trimethylsilyl)amino]-4-[(trimethylsilyl)oxy]-s-triazine. Up to about 20 wt % of the 5-azacytosine and up to about 80 wt % of the HMDS, based on the combined weight of both, can be combined.

Stirring the first intermediate product and heating the first intermediate product to at least about 60° C.; and stirring the first intermediate product while maintaining the first intermediate product at at least about 60° C. for at least about 1.5 hour up to about 24 hours.

Removing at least a portion of, e.g., a substantial portion or substantially all, unreacted HMDS from the first intermediate product. HMDS can be removed by suitable means known to those skilled in the art, for example, HMDS can be removed under high vacuum. Additionally, the first intermediate product can be isolated by forming a slurry of the first intermediate product with a aprotic, non-polar solvent. It is desirable to remove as much HMDS as can be removed. Once the HMDS is removed, the first intermediate product can be handled as a solid, or, upon heating, as a liquid.

Combining at least a substantially anhydrous first Solvent B and at least a portion of the first intermediate product. Commercially available anhydrous acetonitrile typically contains less that about 0.005% water.

Removing at least a portion of the first Solvent B from the first intermediate product. The first Solvent B can be removed by means known to those skilled in the art, such as under vacuum. Substantially all, or all, of the first Solvent B can be removed from the first intermediate product.

Combining at least a portion of the first intermediate product and at least a substantially anhydrous second Solvent B to yield a second intermediate product comprising 2-[(trimethylsilyl)amino]-4-[(trimethylsilyl)oxy]-s-triazine. Up to about a 20% loading of the first intermediate product can be used, e.g., such that up to about 20 grams of the first intermediate product are combined per 100 ml of the second Solvent B.

Combining at least a substantially anhydrous third Solvent B and at least 1-[3,5-di-O-(p-chlorobenzoyl)]-2-deoxy-alpha-D-ribofuranosyl-chloride (e.g., at least a portion of the 1-[3,5-di-O-(p-chlorobenzoyl)]-2-deoxy-alpha-D-ribofuranosyl-chloride produced according to this invention) to yield a first intermediate combination. The first intermediate combination can be either a slurry or a solution. Up to about a 20% loading of the 1-[3,5-di-O-(p-chlorobenzoyl)]-2-deoxy-alpha-D-ribofuranosyl-chloride can be used, e.g., such that up to about 20 grams of the 1-[3,5-di-O-(p-chlorobenzoyl)]-2-deoxy-alpha-D-ribofuranosyl-chloride are combined per 100 ml of the third Solvent B.

Cooling the first intermediate combination to at least about 25° C. to about −10° C. while stirring.

Combining at least the following to yield a second intermediate combination: at least a portion of the first intermediate combination and at least a portion of the fourth second intermediate product.

Combining at least a suitable Catalyst C, a substantially anhydrous fourth Solvent B, and at least a portion of the second intermediate combination to yield a third intermediate combination.

Stirring the third intermediate combination for at least about 2 hours at about −10° C. to about 25° C. in the presence of a second inert gas.

Combining at least the third intermediate combination with a fifth Solvent B to yield a fourth intermediate combination.

Quenching the fourth intermediate combination, e.g., by combining at least the following to form a fifth intermediate combination: (i) either (a) a weak base such as saturated aqueous sodium bicarbonate to adjust the pH of the fourth intermediate combination to about 6 to about 8, or (b) water, methanol, or solid NaHCO₃, (ii) water or brine, and (iii) at least a portion, e.g., substantially all, or all, of the fourth intermediate combination.

Yielding from the fifth intermediate combination at least about 0.8:1 to about 2.7:1 beta:alpha anomers of 4-amino-1-[3,5-di-O-(p-chlorobenzoyl)]-2-deoxy-D-ribofuranosyl-1,3,5-triazin-2(1H)-one, e.g., by concentrating and forming a slurry of the fifth intermediate combination with MeOH or MeOH and hexanes to isolate at least the beta:alpha anomers. Alternatively, by heating the chloroform layer to keep the beta:alpha anomers in solution, otherwise they may gel. The beta:alpha anomers can be isolated as a solid by forming the slurry.

Yielding beta:alpha anomers of 4-amino-1-[3,5-di-O-(p-chlorobenzoyl)]-2-deoxy-D-ribofuranosyl-1,3,5-triazin-2(1H)-one from at least 1-[3,5-di-O-(p-chlorobenzoyl)]-2-deoxy-alpha-D-ribofuranosyl-chloride in accordance with this invention can comprise the following:

Combining at least 5-azacytosine, hexamethyldisalazane (“HMDS”) and a Catalyst B in the presence of an essentially dry first inert gas to yield a first intermediate product comprising 2-[(trimethylsilypamino]-4-[(trimethylsilyl)oxy]-s-triazine. Up to about 20 wt % of the 5-azacytosine and up to about 80 wt % of the HMDS, based on the combined weight of both, can be combined.

Stirring the first intermediate product and heating the first intermediate product to at least about 60° C.; and stirring the first intermediate product while maintaining the first intermediate product at at least about 60° C. for at least about 1.5 hour up to about 24 hours.

Removing at least a portion of, e.g., a substantial portion or substantially all, unreacted HMDS from the first intermediate product. HMDS can be removed by suitable means known to those skilled in the art, for example, HMDS can be removed under high vacuum. Additionally, the first intermediate product can be isolated by forming a slurry of the first intermediate product with a aprotic, non-polar solvent. It is desirable to remove as much HMDS as can be removed. Once the HMDS is removed, the first intermediate product can be handled as a solid, or, upon heating, as a liquid.

Combining at least a substantially anhydrous first Solvent B, at least 1-[3,5-di-O-(p-chlorobenzoyl)]-2-deoxy-alpha-D-ribofuranosyl-chloride (e.g., at least a portion of the 1-[3,5-di-O-(p-chlorobenzoyl)]-2-deoxy-alpha-D-ribofuranosyl-chloride produced according to this invention), and at least a portion of the first intermediate product to yield a first intermediate combination.

Combining at least a Catalyst C, a substantially anhydrous second Solvent B, and at least a portion of the first intermediate combination to yield a second intermediate combination.

Stirring the second intermediate combination at about −10° C. to about 25° C. for at least about 2 hours.

Combining at least a weak base and the second intermediate combination to adjust the pH of the second intermediate combination to about 6 to about 8. Suitable weak bases can comprise sodium bicarbonate, e.g., saturated aqueous sodium bicarbonate, Na₂CO₃, or the like.

Yielding from the second intermediate combination at least a composition comprising one or more organic compounds, e.g., by performing a phase cut. The composition can be washed with, e.g., brine or water and dried, e.g., over magnesium sulfate or molecular sieves or sodium sulfate or other suitable drying agents.

Yielding from the composition at least beta:alpha anomers of 4-amino-1-[3,5-di-O-(p-chlorobenzoyl)]-2-deoxy-D-ribofuranosyl-1,3,5-triazin-2(1H)-one.

### Yielding Decitabine From At Least the Beta:Alpha Anomers of 4-amino-1-[3,5-di-O-(p-chlorobenzoyl)]-2-deoxy-D-ribofuranosyl-1,3,5-triazin-2(1H)-one

Yielding decitabine from at least beta:alpha anomers of 4-amino-1-[3,5-di-O-(p-chlorobenzoyl)]-2-deoxy-D-ribofuranosyl-1,3,5-triazin-2(1H)-one in accordance with this invention can comprise the following:

Combining at least substantially anhydrous methanol, an inert gas, and at least a portion of the beta:alpha anomers of 4-amino-1-[3,5-di-O-(p-chlorobenzoyl)]-2-deoxy-D-ribofuranosyl-1,3,5-triazin-2(1H)-one at ambient temperature to yield a penultimate combination.

Combining at least NaOMe in methanol and at least a portion of the penultimate combination while stirring to yield a final combination and continuing the stirring of the final combination for about 1 to about 4 hours at ambient temperature, then cooling to about 25° C. to about 0° C. and maintaining at about 25° C. to about 0° C. for about 30 minutes to about 60 minutes.

Yielding a solid from the final combination.

Rinsing the solid with a substantially anhydrous final rinse solvent e.g., with substantially anhydrous methanol.

Optionally, drying the solid to yield decitabine.

### Yielding Decitabine From At Least the Beta:Alpha Anomers of 4-amino-1-[3,5-di-O-(p-chlorobenzoyl)]-2-deoxv-D-ribofuranosyl-1,3,5-triazin-2(1H)-one

Yielding decitabine from at least beta:alpha anomers of 4-amino-1-[3,5-di-O-(p-chlorobenzoyl)]-2-deoxy-D-ribofuranosyl-1,3,5-triazin-2(1H)-one in accordance with this invention can comprise the following:

Combining at least substantially anhydrous methanol, an inert gas, and at least a portion of the beta:alpha anomers of 4-amino-1-[3,5-di-O-(p-chlorobenzoyl)]-2-deoxy-D-ribofuranosyl-1,3,5-triazin-2(1H)-one at ambient temperature to yield a penultimate combination.

Combining at least NaOMe in methanol and at least a portion of the penultimate combination while stirring to yield a final combination and continuing the stirring of the final combination for about 1 to about 4 hours at ambient temperature, then cooling to about 25° C. to about 0° C. and maintaining at about 25° C. to about 0° C. for about 30 minutes to about 60 minutes.

Yielding a solid from the final combination, e.g., via vacuum filtration and rinsing the solid with a suitable final rinse solvent, then drying the collected solid, e.g., under high vacuum, to yield decitabine.

### EXAMPLES

The following examples are illustrative of the principles of this invention. It is understood that this invention is not limited to any one specific embodiment exemplified herein, whether in the examples or the remainder of this patent application.

### Example 1

### Preparation of 1-[3,5-di-O-(p-chlorobenzoyl)]-2-deoxy-alpha-D-ribofuranosyl-chloride

Into a 4-neck 3 L RB flask that was dried under a nitrogen stream was charged 100 g of 2-deoxy-D-ribose and anhydrous methanol (500 mL). The mixture was cooled to 7° C. Under mixing, acetyl chloride (1.56 g) was added through a syringe. After 1.5 hr at this temperature triethylamine (4.24 g) was added and mixing was continued for 10 min. to yield 1-O-methyl-2-deoxy-D-ribofuranose. Methanol was removed under vacuum using a warm water bath (40° C.). Residual methanol was removed by adding dioxane (189 g) then removing the residual methanol under vacuum using a warm water bath. Into the 1-O-methyl-2-deoxy-D-ribofuranose was added dioxane (1.8 L), 4-dimethylaminopyridine (DMAP, 3 g), and triethylamine (171 g). Then p-chlorobenzoyl chloride (268.5 g) was added slowly while the temperature was maintained at 15-18° C. No aqueous work-up was required. The slurry was mixed at room temperature overnight then vacuum filtered into a 5-L 4-neck RB flask. The cake was rinsed with dioxane (0.2 L); and the rinse was combined with the filtrate. Acetic acid was added (0.84 kg) to the rinse/filtrate mixture, followed by the addition of acetyl chloride (14.1 g). HCl gas (330 g) was fed while maintaining temperature between 12-18° C. Precipitate started to form and the slurry was mixed for an additional 20 minutes after HCl addition was complete. Hexane was added (0.5 L) and the product was collected via vacuum filtration. After rinsing with hexane (0.3 L) the product was vacuum-dried to give 144 g of 1-[3,5-di-O-(p-chlorobenzoyl)]-2-deoxy-alpha-D-ribofuranosyl-chloride as white solid for use in Examples 4 and 5.

### Example 2

### Preparation of 1-[3,5-di-O-(p-chlorobenzoyl)]-2-deoxy-alpha-D-ribofuranosyl-chloride.

1-O-methyl-3,5-di-O-p-chlorobenzoyl-2-deoxy-alpha/beta-D-ribose (15.94 g), glacial acetic acid (60 mL), anhydrous chloroform (60 mL) and acetyl chloride (0.85 g) were added into a 4-neck, 250 mL round-bottom flask. Under stirring and nitrogen gaseous, HCl was fed (6.5 g) subsurface while the temperature was maintained between 13 to 15° C. 1-[3,5-di-O-(p-chlorobenzoyl)]-2-deoxy-alpha-D-ribofuranosyl-chloride slowly crystallized out during HCl feeding. The slurry was stirred at this temperature for 15 minutes after HCl feeding was complete. The product was collected and rinsed with anhydrous hexanes (20 mL). After drying, 10.9 g of 1-[3,5-di-O-(p-chlorobenzoyl)]-2-deoxy-alpha-D-ribofuranosyl-chloride was isolated as white powder (69% yield) for use in Example 6.

### Example 3

### Preparation of 1-[3,5-di-O-(p-chlorobenzoyl)]-2-deoxy-alpha-D-ribofuranosyl-chloride.

1-O-methyl-3,5-di-O-p-chlorobenzoyl-2-deoxy-alphalbeta-D-ribose (15.94 g), glacial acetic acid (60 mL), anhydrous chloroform (30 mL), anhydrous dioxane (30 mL) and acetyl chloride (0.87 g) were added into a 4-neck, 250 mL round-bottom flask. Under stirring and nitrogen, gaseous HCl was fed (11.5 g) subsurface while the temperature was maintained between 13 to 15° C. 1-[3,5-di-O-(p-chlorobenzoyl)]-2-deoxy-alpha-D-ribofuranosyl-chloride slowly crystallized out during HCl feeding. The slurry was stirred at 12° C. for 20 minutes after HCl feeding was complete. The product was collected and rinsed with anhydrous hexanes (20 mL). After drying, 12.4 g of 1-[3,5-di-O-(p-chlorobenzoyl)]-2-deoxy-alpha-D-ribofuranosyl-chloride was isolated as white powder (77% yield).

### Example 4

### Preparation of 4-amino-1-[3,5-di-O-(p-chlorobenzoyl)]-2-deoxy-alpha-D-ribofuranosyl-1,3,5-triazin-2(1H)-one and 4-amino-1-[3,5-di-O-(p-chlorobenzoyl)]-2-deoxy-beta-D-ribofuranosyl-1,3,5-triazin-2(1H)-one.

Into a 4-neck 1 L RB flask was charged 5-azacytosine (6.12 g, 0.055 mol), hexamethyldisalazane (HMDS, 300 mL) and chlorotrimethylsilane (6.8 mL) under nitrogen. The mixture was heated to 117° C. with mixing and was held at this temperature for 5 hr. The excess HMDS was removed under high vacuum and anhydrous acetonitrile was (100 mL) added. The solvent was removed under vacuum and was dissolved in acetonitrile (150 mL) to give 4-amino-1-[3,5-di-O-(p-chlorobenzoyl)]-2-deoxy-D-ribofuranosy1-1,3,5-triazin-2(1H)-one.

Into a dried 1 L 4-neck RB flask was charged 1-[3,5-di-O-(p-chlorobenzoyl)]-2-deoxy-alpha-D-ribofuranosyl-chloride (9.3 g, 21.7 mmol, from Example 1) and anhydrous acetonitrile (150 mL). The slurry was cooled to 1° C. under mixing and into this was charged the 4-amino-1-[3,5-di-O-(p-chlorobenzoyl)]-2-deoxy-D-ribofuranosyl-1,3,5-triazin-2(1H)-one solution followed by the addition of trimethylsilyl trifluoromethanesulfonate (1.99 g, 9.0 mmol) in acetonitrile (15 mL). The mixture was mixed for 26 hrs at 1° C. under nitrogen then was diluted with chloroform (500 mL). The reaction was quenched by the addition of brine (100 mL) and saturated sodium bicarbonate (20 mL). After phase separation, the organic phase was washed with brine (1×150 mL, 1×100 mL) then dried over MgSO₄. After filtration through a bed of celite under vacuum, the filtrate was concentrated to dryness on rotavap. After azeotroping with toluene (300 mL), an off-white solid (9.67 g) was obtained which contains 1.45:1 of beta:alpha anomers of 4-amino-1-[3,5-di-O-(p-chlorobenzoyl)]-2-deoxy-D-ribofuranosyl-1,3,5-triazin-2(1H)-one. It is ready to be used in Example 7 without further purification.

### Example 5

### Preparation of 4-amino-1-[3,5-di-O-(p-chlorobenzoyl)]-2-deoxy-alpha-D-ribofuranosyl-1,3,5-triazin-2(1H)-one and 4-amino-1-[3,5-di-O-(p-chlorobenzoyl)]-2-deoxy-beta-D-ribofuranosyl-1,3,5-triazin-2(1H)-one.

Into a 4-neck 1 L RB flask was charged 5-azacytosine (5.23 g, 0.047 mol), hexamethyldisalazane (HMDS, 200 mL) and chlorotrimethylsilane (2 mL) under nitrogen. The mixture was heated to 117° C. with mixing and was held at this temperature for 5 hr. The excess HMDS was removed under high vacuum and anhydrous chloroform was (100 mL) added. The solvent was removed under vacuum and was dissolved in chloroform (150 mL) to give 4-amino-1-[3,5-di-O-(p-chlorobenzoyl)]-2-deoxy-D-ribofuranosyl-1,3,5-triazin-2(1H)-one.

Into a dried 1 L 4-neck RB flask was charged 1-[3,5-di-O-(p-chlorobenzoyl)]-2-deoxy-alpha-D-ribofuranosyl-chloride (9.3 g, 21.7 mmol, from Example 1) and anhydrous chloroform (150 mL). The slurry was cooled to 1° C. under mixing and into this was charged the 4-amino-1-[3,5-di-O-(p-chlorobenzoyl)]-2-deoxy-D-ribofuranosyl-1,3,5-triazin-2(1H)-one solution followed by the addition of trimethylsilyl trifluoromethanesulfonate (1.99 g, 9.0 mmol) in chloroform (17 mL). The mixture was mixed for 44 hr at 1° C. under nitrogen. The reaction was quenched by the addition of brine (100 mL) and saturated sodium bicarbonate (40 mL). After phase separation, the organic phase was washed with brine (2×100 ml) then dried over MgSO₄. After filtration through a bed of celite under vacuum, the filtrate was concentrated to dryness on rotavap. After azeotroping with chloroform and toluene, a off-white solid (29.82 g) was obtained which contains 29 wt % of the 4-amino-1-[3,5-di-O-(p-chlorobenzoyl)]-2-deoxy-beta-D-ribofuranosyl-1,3,5-triazin-2(1H)-one and 14 wt % of the 4-amino-1-[3,5-di-O-(p-chlorobenzoyl)]-2-deoxy-alpha-D-ribofuranosyl-1,3,5-triazin-2(1H)-one. This material is ready to be used in Example 8 without further purification.

### Example 6

### Preparation of 4-amino-1-[3,5-di-O-(p-chlorobenzoyl)]-2-deoxy-alpha-D-ribofuranosyl-1,3,5-triazin-2(1H)-one and 4-amino-1-[3,5-di-O-(p-chlorobenzoyl)]-2-deoxy-beta-D-ribofuranosyl-1,3,5-triazin-2(1H)-one.

Purified 5-azacytosine (1.62 g), ammonium sulfate (0.162 g) and hexamethyldisilazane (40 mL) were charged into a 4-neck, 100 mL round-bottom flask under nitrogen. The mixture was heated to 117° C. under stirring and kept at this temperature for 2 hours to give a clear solution. Unreacted hexamethyldisilazane was removed under vacuum to give 2-[(trimethylsilyl)amino]-4-[(trimethylsilyl)oxy]-s-triazine as a white solid. The solid was dissolved in anhydrous chloroform (25 mL) and was added into a solution of 1-[3,5-di-O-(p-chlorobenzoyl)]-2-deoxy-alpha-D-ribofuranosyl-chloride (5.0 g, from Example 2) in anhydrous chloroform and stirred at 1° C. under nitrogen. Trimethylsilyl trifluoromethanesulfonate (1.0 g) in anhydrous chloroform (10 mL) was added and the resulting mixture was stirred at this temperature for 21 hours. Saturated aqueous sodium bicarbonate was added to neutralize to pH 6. The mixture was diluted with additional chloroform (100 mL). After phase cut, the organic layer was washed with brine (30 mL) and dried over magnesium sulfate. The mixture was filtered through a bed of filter aid. Methanol was added and the solution was concentrated to 20 mL followed by the addition of anhydrous hexanes (40 mL) under stirring to form slurry. The solid was collected and dried under vacuum to give the product (5.06 g) that contains a mixture of 2.5:1 beta:alpha anomers. It is ready to be used in a process according to this invention for preparing Decitabine, without further purification.

### Example 7

### Preparation of Decitabine

Into a dry 4-neck 100 mL RB flask was charged the 4-amino-1-[3,5-di-O-(p-chlorobenzoyl)]-2-deoxy-alpha-D-ribofuranosyl-1,3,5-triazin-2(1H)-one and 4-amino-1-[3,5-di-O-(p-chlorobenzoyl)]-2-deoxy-beta-D-ribofuranosyl-1,3,5-triazin-2(1H)-one product (3.44 g, from Example 4) and anhydrous methanol under nitrogen and at room temperature. While mixing, 25 wt % NaOMe in methanol (1.42 g) was added. The slurry was mixed for 4 hours at room temperature then cooled to 3° C. and held for 30 min. The solid formed was collected via vacuum filtration and was rinsed with anhydrous methanol (3 mL). The product was dried under high vacuum to give decitabine as white solid (0.36 g). HPLC analysis indicated 98.9% purity. Although not done, the purity of this product can be improved by treatment with charcoal and recrystallization from anhydrous methanol.

### Example 8

### Preparation of Decitabine

Into a dry 4-neck 1L RB flask was charged crude 4-amino-1-[3,5-di-O-(p-chlorobenzoyl)]-2-deoxy-alpha-D-ribofuranosyl-1,3,5-triazin-2(1H)-one and 4-amino-1-[3,5-di-O-(p-chlorobenzoyl)]-2-deoxy-beta-D-ribofuranosyl-1,3,5-triazin-2(1H)-one product (25 g, from Example 5) and anhydrous methanol (300 mL) under nitrogen and at room temperature. While mixing, 25 wt % NaOMe in methanol (5.41 g) was added. The slurry was mixed for 4 hours at room temperature then cooled to 3° C. and held for 30 min. The solid formed was collected via vacuum filtration and was rinsed with anhydrous methanol (20 mL). The product was dried under high vacuum to give crude decitabine as white solid (1.95 g). This crude decitabine was treated with charcoal and recrystallized from anhydrous methanol to give 0.92 g of decitabine. HPLC analysis indicated 99.9% purity.

It is to be understood that the reactants and components referred to by chemical name or formula anywhere in the specification or claims hereof, whether referred to in the singular or plural, are identified as they exist prior to being combined with or coming into contact with another substance referred to by chemical name or chemical type (e.g., another reactant, a solvent, or etc.). It matters not what chemical changes, transformations and/or reactions, if any, take place in the resulting combination or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical reaction or in forming a combination to be used in conducting a desired reaction. Accordingly, even though the claims hereinafter may refer to substances, components and/or ingredients in the present tense (“comprises”, “is”, etc.), the reference is to the substance, component or ingredient as it existed at the time just before it was first contacted, combined, blended or mixed with one or more other substances, components and/or ingredients in accordance with the present disclosure. Whatever transformations, if any, which occur in situ as a reaction is conducted is what the claim is intended to cover. Thus the fact that a substance, component or ingredient may have lost its original identity through a chemical reaction or transformation during the course of contacting, combining, blending or mixing operations, if conducted in accordance with this disclosure and with the application of common sense and the ordinary skill of a chemist, is thus wholly immaterial for an accurate understanding and appreciation of the true meaning and substance of this disclosure and the claims thereof. As will be familiar to those skilled in the art, the terms “combined”, “combining”, “added”, “adding” and the like as used herein mean that the components that are “combined” or that one is “combining” are put into a container with each other. Likewise a “combination” of components means the components having been put together in a container.

While the present invention has been described in terms of one or more preferred embodiments, it is to be understood that other modifications may be made without departing from the scope of the invention, which is set forth in, the claims below.

What is claimed is:

1. A process comprising:
(a) combining at least 2-deoxy-D-ribose and methanol to yield a first combination, wherein the amount of methanol is at least about 4 grams per gram of the 2-deoxy-D-ribose and the methanol is substantially anhydrous;
(b) cooling the first combination to about 25° C. to about −20° C.;
(c) combining at least an acyl halide and at least a portion of the first combination to yield a second combination;
(d) maintaining the second combination at about 25° C. to about −20° C. for about 30 minutes to about 240 minutes;
(e) combining at least a first volatile tertiary amine and at least a portion of the second combination to yield a first product comprising 1-O-methyl-2-deoxy-D-ribofuranose and methanol;
(f) removing substantially all of the methanol from the first product, yielding a substantially methanol-free first product;
(g) combining at least a substantially anhydrous Solvent A, a Catalyst A, a second volatile tertiary amine, and at least a portion of the substantially methanol-free first product to yield a third combination;
(h) combining at least p-chlorobenzoyl chloride and at least a portion of the third combination to yield a fourth combination;
(i) stirring the fourth combination for about 2 hours to about 6 hours at about 10° C. to about 40° C.;
(j) filtering the fourth combination to yield a filtrate and a filter cake;
(k) collecting the filtrate;
(l) rinsing the filter cake with at least a substantially anhydrous rinsing solvent;
(m) combining at least acetic acid, at least a portion of the filtrate, and at least a portion of the used rinsing solvent to yield a fifth combination;
(n) combining at least HCl gas and at least a portion of the fifth combination at about −10° C. to about 25° C. to yield a sixth combination;
(o) stirring the sixth combination; (p) collecting a second product comprising 1-[3,5-di-O-(p-chlorobenzoyl)]-2-deoxy-alpha-D-ribofuranosyl-chloride from the sixth combination;
(q) drying the second product to yield at least 1-[3,5-di-O-(p-chlorobenzoyl)]-2-deoxy-alpha-D-ribofuranosyl-chloride;
(r) yielding beta:alpha anomers of 4-amino-1-[3,5-di-O-(p-chlorobenzoyl)]-2-deoxy-D-ribofuranosyl-1,3,5-triazin-2(1H)-one from at least the 1-[3,5-di-O-(p-chlorobenzoyl)]-2-deoxy-alpha-D-ribofuranosyl-chloride; and
(s) yielding decitabine from at least the beta:alpha anomers of 4-amino-1-[3,5-di-O-(p-chlorobenzoyl)]-2-deoxy-D-ribofuranosyl-1,3,5-triazin-2(1H)-one.

2. A process comprising:
(a) combining at least 1-O-methyl-3,5-di-O-p-chlorobenzoyl-2-deoxy-alpha/beta-D-ribose, acetic acid, an acyl halide, and a substantially anhydrous solvent to yield a first combination;
(b) while stirring and in the presence of an inert gas, feeding gaseous HCl to the first combination at about −10° C. to about 25° C. to yield a second combination comprising 1-[3,5-di-O-(p-chlorobenzoyl)]-2-deoxy-alpha-D-ribofuranosyl-chloride;
(c) stirring the second combination at about −10° C. to about 25° C.;
(d) yielding from the second combination a product comprising 1-[3,5-di-O-(p-chlorobenzoyl)]-2-deoxy-alpha-D-ribofuranosyl-chloride;
(e) rinsing the product with a rinse;
(f) drying the product to yield at least 1-[3,5-di-O-(p-chlorobenzoyl)]-2-deoxy-alpha-D-ribofuranosyl-chloride;
(g) yielding beta:alpha anomers of 4-amino-1-[3,5-di-O-(p-chlorobenzoyl)]-2-deoxy-D-ribofuranosyl-1,3,5-triazin-2(1H)-one from at least the 1-[3,5-di-O-(p-chlorobenzoyl)]-2-deoxy-alpha-D-ribofuranosyl-chloride; and
(h) yielding decitabine from at least the beta:alpha anomers of 4-amino-1-[3,5-di-O-(p-chlorobenzoyl)]-2-deoxy-D-ribofuranosyl-1,3,5-triazin-2(1H)-one.

3. A process according to claim 1 or 2, wherein yielding beta:alpha anomers of 4-amino-1-[3,5-di-O-(p-chlorobenzoyl)]-2-deoxy-D-ribofuranosyl-1,3,5-triazin-2(1H)-one from at least the 1-[3,5-di-O-(p-chlorobenzoyl)]-2-deoxy-alpha-D-ribofuranosyl-chloride comprises:
(a) combining at least 5-azacytosine, hexamethyldisalazane and a Catalyst B in the presence of a first inert gas to yield a first intermediate product comprising 2-[(trimethylsilypamino]-4-[(trimethylsilyl)oxy]-s-triazine;
(b) stirring and heating the first intermediate product to at least about 60° C.;
(c) stirring the first intermediate product while maintaining the first intermediate product at at least about 60° C.;
(d) removing at least a portion of unreacted hexamethyldisalazane from the first intermediate product;
(e) combining at least a substantially anhydrous first Solvent B and at least a portion of the first intermediate product;
(f) removing at least a portion of the first Solvent B from the first intermediate product;
(g) combining at least a portion of the first intermediate product and at least a substantially anhydrous second Solvent B to yield a second intermediate product comprising 2-[(trimethylsilypamino]-4-[(trimethylsilypoxy]-s-triazine,
(h) combining at least a substantially anhydrous third Solvent B and at least a portion of the 1-[3,5-di-O-(p-chlorobenzoyl)]-2-deoxy-alpha-D-ribofuranosyl-chloride to yield a first intermediate combination;
(i) cooling the first intermediate combination to at least about 25° C. to about −10° C. while stirring;
(j) combining at least the following to yield a second intermediate combination: at least a portion of the first intermediate combination and at least a portion of the second intermediate product;
(k) combining at least a Catalyst C, a substantially anhydrous fourth Solvent B, and at least a portion of the second intermediate combination to yield a third intermediate combination;
(l) stirring the third intermediate combination at about −10° C. to about 25° C. in the presence of a second inert gas;
(m) combining at least the third intermediate combination and a fifth Solvent B to yield a fourth intermediate combination;
(n) combining at least the following to form a fifth intermediate combination:
(i) either (a) a weak base such as saturated aqueous sodium bicarbonate to adjust the pH of the fourth intermediate combination to about 6 to about 8, or (b) water, methanol, or solid NaHCO₃, (ii) water or brine, and (iii) at least a portion, e.g., substantially all, or all, of the fourth intermediate combination; and
(o) yielding at least beta:alpha anomers of 4-amino-1-[3,5-di-O-(p-chlorobenzoyl)]-2-deoxy-D-ribofuranosyl-1,3,5-triazin-2(1 H)-one from the fifth intermediate combination.

4. A process according to claim 1 or 2, wherein yielding beta:alpha anomers of 4-amino-1-[3,5-di-O-(p-chlorobenzoyl)]-2-deoxy-D-ribofuranosyl-1,3,5-triazin-2(1H)-one from at least the 1-[3,5-di-O-(p-chlorobenzoyl)]-2-deoxy-alpha-D-ribofuranosyl-chloride comprises:
(a) combining at least 5-azacytosine, hexamethyldisalazane and a Catalyst B in the presence of a first inert gas to yield a first intermediate product comprising 2-[(trimethylsilypamino]-4-[(trimethylsilyl)oxy]-s-triazine;
(b) stirring and heating the first intermediate product to at least about 60° C.;
(c) stirring the first intermediate product while maintaining the first intermediate product at at least about 60° C.;
(d) removing at least a portion of unreacted hexamethyldisalazane from the first intermediate product;
(e) combining at least a substantially anhydrous first Solvent B, at least a portion of the 1-[3,5-di-O-(p-chlorobenzoyl)]-2-deoxy-alpha-D-ribofuranosyl-chloride, and at least a portion of the first intermediate product to yield a first intermediate combination;
(f) combining at least a Catalyst C, a substantially anhydrous second Solvent B, and at least a portion of the first intermediate combination to yield a second intermediate combination;
(g) stirring the second intermediate combination at about −10° C. to about 25° C.;
(h) combining at least a weak base and the second intermediate combination to adjust the pH of the second intermediate combination to about 6 to about 8;
(i) yielding from the second intermediate combination at least a composition comprising one or more organic compounds; and
(j) yielding from the composition at least beta:alpha anomers of 4-amino-1-[3,5-di-O-(p-chlorobenzoyl)]-2-deoxy-D-ribofuranosyl-1,3,5-triazin-2(1H)-one.

5. A process according to claim 1 or 2 wherein yielding decitabine from at least the beta:alpha anomers of 4-amino-1-[3,5-di-O-(p-chlorobenzoyl)]-2-deoxy-D-ribofuranosyl-1,3,5-triazin-2(1H)-one comprises:
(a) combining at least substantially anhydrous methanol, an inert gas, and at least a portion of the beta:alpha anomers of 4-amino-1-[3,5-di-O-(p-chlorobenzoyl)]-2-deoxy-D-ribofuranosyl-1,3,5-triazin-2(1H)-one to yield a penultimate combination;
(b) combining at least NaOMe in methanol and at least a portion of the penultimate combination while stirring to yield a final combination;
(c) yielding a solid from the final combination; and
(d) rinsing the solid with a substantially anhydrous final rinse solvent to yield decitabine.

6. A process according to claim 1 or 2 wherein yielding decitabine from at least the beta:alpha anomers of 4-amino-1-[3,5-di-O-(p-chlorobenzoyl)]-2-deoxy-D-ribofuranosyl-1,3,5-triazin-2(1H)-one comprises:
(a) combining at least substantially anhydrous methanol, an inert gas, and at least a portion of the beta:alpha anomers of 4-amino-1-[3,5-di-O-(p-chlorobenzoyl)]-2-deoxy-D-ribofuranosyl-1,3,5-triazin-2(1H)-one to yield a penultimate combination;
(b) combining at least NaOMe in methanol and at least a portion of the penultimate combination while stirring to yield a final combination;
(c) stirring the final combination for about 1 to about 4 hours at ambient temperature;
(d) cooling the final combination to about 25° C. to about 0° C. and maintaining at about 25° C. to about 0° C. for about 30 minutes to about 60 minutes;
(e) yielding a solid from the final combination;
(f) rinsing the solid with a final rinse solvent; and
(g) drying the solid to yield decitabine.
